(19) United States
(12) Patent Application Publication
(10) Pub. No.: US 2024/0000807 A1
(43) Pub. Date: Jan. 4, 2024

(54) DRY SELF-EMULSIFYING CANNABINOID COMPOSITIONS AND USE THEREOF

(71) Applicant: Technology Holding Company LLC, Waukesha, WI (US)

(72) Inventor: Jeremy James Smith, Waukesha, WI (US)

(21) Appl. No.: 18/215,458

(22) Filed: Jun. 28, 2023

Related U.S. Application Data
(60) Provisional application No. 63/356,263, filed on Jun. 28, 2022.

(51) Int. Cl. A61K 31/00; A61K 47/54; A61K 9/00

(52) U.S. Cl. CPC A61K 31/658; A61K 47/54; A61K 9/0095; A61K 9/0056

(57) ABSTRACT

Provided herein are compositions comprising cannabinoid cyclodextrin inclusion complex compressed with effervescing agents, and having enhanced dissolution, dispersion and shelf-life, as well as methods of use thereof. The methods include methods of producing cannabinoid inclusion complexes dispersed in solutions, and method of making cannabinoid-infused food products or beverages using the self-emulsifying compositions of cannabinoid cyclodextrin inclusion complex.

### BACKGROUND

The use of cannabis for recreational and medical purposes is legal in many countries under specific cannabis legislation. The most prevalent mode of use of cannabis is by smoking, however, conventional smoking has adverse effects on the lungs due to the harmful inhalants released when burning plant matter. Smoke contains tar and particulate matter which can contribute to lung diseases including lung cancer and can include health risks such as irritation to the mouth, esophagus, and lungs. Because inhaled cannabis is short-acting it may need to be smoked several times a day thus increasing the health risks of smoke inhalation.

Alternate inhalation methods such as electronic smoking devices offer significant health benefits over conventional smoking products, however, such inhalation methods can still be unacceptable to non-smokers. Additionally, the general unappealing nature of smoking or vaping and an increased focus on public health such as banning smoking in public places has restricted the convenience of administration of cannabinoids by smoking and vaping methods.

Cannabis compositions developed for edible use overcome the health risks and social challenges associated with smoking and vaporization methods. Cannabinoid edibles make up a significant proportion of total cannabinoid use globally, however, a considerable percentage of edible cannabinoid products are not adapted for use in casual recreational and social settings, or routine retail sales to consumers.

There is an unmet need for cannabinoid dosage forms that can uniformly disperse cannabinoids into any aqueous beverage, for example with minimal mixing and controlled dosing, in stable compositions, and a consumer-friendly dosage form. Such products would enable on-demand beverage preparation for the administration of controlled amounts of cannabinoids in a single delivery form. Such a product should uniformly infuse any beverage composition with a cannabinoid without influencing taste and in a convenient way that also notifies the user that an active ingredient was introduced to the drink. Additionally, such product should be acceptable for use in social gatherings without compromising public health policies.

### SUMMARY

The present disclosure is based, in part, on the development of self-emulsifying cannabinoid compositions having enhanced dispersion properties, and methods of use thereof for producing cannabinoid inclusion complexes dispersed in a solution and for making a cannabinoid-infused food product or beverage. More particularly, the present disclosure relates to compositions and dosage forms of cannabinoids and cannabinoid related compounds, effervescing agents, and emulsifiers, which quickly self-emulsify to generate a uniform solution upon contact with water and produces a high concentration of dissolved cannabinoids, thereby possessing enhanced emulsification efficiency.

An embodiment provides a composition comprising: (i) from about 20 to 95 wt % of an effervescing agent (ii) from about 5 to 80 wt % of a cannabinoid cyclodextrin inclusion complex and (iii) from about 0 to % 5 wt % of an emulsifier. In some embodiments, the amount of the effervescent agent in the composition is about 20-95 wt %, 25-95 wt %, 30-95 wt %, 35-95 wt %, 40-95 wt %, 45-95 wt %, 50-95 wt %, 55-95 wt %, 60-95 wt %, 65-95 wt %, 70-95 wt %, 75-95 wt %, 80-95 wt %, 85-95 wt %, 20-85 wt %, 25-85 wt %, 30-85 wt %, 35-85 wt %, 40-85 wt %, 45-85 wt %, 50-85 wt %, 55-85 wt %, 60-85 wt %, 65-85 wt %, 70-85 wt %, 75-85 wt %, or 80-85 wt %. In some embodiments, the amount of the cannabinoid cyclodextrin inclusion complex in the composition is 5 to 70%, 5 to 60%, 5-50 wt %, 5-40 wt %, 5-30 wt %, 5-20 wt %, 10-80 wt %, 10-70 wt %, 10-60 wt %, 10-50 wt %, 10-40 wt %, 10-30 wt %, 10-20 wt %, 10-15 wt %, or 15-20 wt %. In some embodiments, the amount of the emulsifier in the composition is 0-4 wt %, 0-3 wt %, 0-2 wt %, 0.05-5 wt %, 0.05-4 wt %, 0.05-3 wt %, 0.05-2 wt %, 0.5-5 wt %, 0.5-4 wt %, 0.5-3 wt %, or 0.5-2 wt %.

The cannabinoid cyclodextrin inclusion complex can comprise a cyclodextrin host and a cannabinoid guest. The effervescing agent can comprise an acidic agent and an alkaline agent. The acidic agent can be citric acid, ascorbic acid, or a combination thereof. The alkaline agent can be a sodium salt of bicarbonate, a potassium salt of bicarbonate, a sodium salt of carbonate, a potassium salt of carbonate or any combination thereof. In some embodiments, the relative acidic agent:alkaline agent ratio (weight ratio) is from 10:1 to 1:10, e.g., from 9:1 to 1:9, from 8:1 to 1:8. from 7:1 to 1:7, from 6:1 to 1:6, from 5:1 to 1:5, from 4:1 to 1:4, from 3:1 to 1:3, from 2:1 to 1:2, from 1.5:1 to 1:1.5, or from 1.3:1 to 1:1.3. The cannabinoid guest can be selected from the group consisting of delta-8 tetrahydrocannabinol (THC), delta-9 THC, delta-10 THC, cannabidiol (CBD), cannabinol (CBN), cannabigerol (CBG), hexahydrocannibinol (HHC), tetrahydrocannabivarin (THCV), tetrahydrocannabiphorol (THCP), O-acetate thereof, isomers thereof, and combination thereof. The cyclodextrin host can be alpha-cyclodextrin, beta-cyclodextrin, gamma-cyclodextrin, 2-Hydroxypropyl-β-cyclodextrin, β-cyclodextrin sulfobutyl ether, or combinations thereof. The composition can further comprise one or more additives. The composition can comprise about 5-25 wt % of the one or more additives. The emulsifier can be a surfactant, The surfactant can be a natural emulsifier or polysorbate 80. The one or more additives can be selected from the group consisting of terpenes, terpenoids, flavonoids, excipients, antioxidants, sweeteners, lubricants, colorants, vitamins, disintegrants, defoamers, foaming agents, wax, bitter blockers, and combinations thereof. The cannabinoid cyclodextrin inclusion complex can comprise about 5-25 wt % cannabinoid and about 75-95 wt % cyclodextrin. In some embodiments, the cannabinoid cyclodextrin inclusion complex comprises about 10-20 wt % cannabinoid and about 80-90 wt % cyclodextrin. In some embodiments, the cannabinoid cyclodextrin inclusion complex comprises about 15-20 wt % cannabinoid and about 80-85 wt % cyclodextrin. In certain embodiments, the cannabinoid cyclodextrin inclusion complex comprises about 16 wt % of cannabinoid and about 84 wt % cyclodextrin. The composition can have a dispersibility ranging from about 50 to 95%. The composition can have a shelf-life of at least about 45 days. The composition can have at least 2-times more dispersity than a control composition that does not comprise a surfactant. The composition retains at least 2 times the concentration of cannabinoid in solution. The composition can be formulated as a powder, an effervescent powder, a tablet, an effervescent tablet, granules or effervescent granules.

In some embodiments, the composition comprises: (i) about 30-36 wt % sodium bicarbonate, (ii) about 1-6 wt % potassium carbonate, (iii) about 40-50 wt % citric acid, (iv) about 10-18 wt % ß-cyclodextrin, (v) about 1-5 wt % delta-8 THC, and (vi) from about 0 to 5 wt % (e.g., 0.05 to 5 wt %) Polysorbate 80. In some embodiments, the composition comprises: (i) about 32 wt % sodium bicarbonate, (ii) about 3 wt % potassium carbonate, (iii) about 45 wt % citric acid, (iv) about 14 wt % ß-cyclodextrin, (v) about 2.5 wt % delta-8 THC, and (vi) from about 0 to 2 wt % (e.g., 0.05 to 2 wt %) Polysorbate 80.

Another embodiment provides a method of producing cannabinoid inclusion complexes dispersed in a solution comprising contacting any one of the compositions described herein with an aqueous solution

The cannabinoid inclusion complexes dispersed solution can comprise at least 50% cannabinoid inclusion complexes. Contacting the composition and the aqueous solution can be without agitation or shaking. Contacting the composition and the aqueous solution can comprise contacting at a temperature as low as 1° C.

An additional embodiment provides a method of making a cannabinoid-infused food product or beverage comprising: (i) producing cannabinoid inclusion complexes dispersed in a solution by contacting any one of the compositions described herein with an aqueous solution, and (ii) incorporating the cannabinoid inclusion complexes dispersed in a solution of (i) in a food product or in a beverage, thereby making a cannabinoid-infused food product or beverage.

The food product or beverage can be selected from the group consisting of jellies, edibles, non-alcoholic beverages, and alcoholic beverages.

### BRIEF DESCRIPTION OF THE DRAWINGS

The accompanying drawings are included to provide a further understanding of the methods and compositions of the disclosure, are incorporated in, and constitute a part of this specification. The drawings illustrate one or more embodiments of the disclosure, and together with the description serve to explain the concepts and operation of the disclosure.

FIG. 1 is a graph illustrating the absorption spectra for delta-8 THC cyclodextrin inclusion complex dispersed in various ethanol/water cosolvents.

FIG. 2 is a graph illustrating the Lambda max signal for various ethanol/water mixtures.

FIG. 3 is graph illustrating UV/Vis spectra obtained 0, 6.25, 12.5, 25, 50, and 100 ppm delta-8 THC concentrations.

FIG. 4 is a graph illustrating 6-point calibration curve obtained from UV/Vis spectra from solutions of beta-cyclodextrin complexes of delta-8 THC.

FIG. 5 is a bar graph illustrating the percent of added beta-cyclodextrin inclusion complex dispersed in solution from an effervescent composition and the resulting stability of solutions over 45 days.

FIG. 6 is a bar graph illustrating the percent of added delta-8 THC dispersed in solution from effervescent compositions with varying concentrations of polysorbate 80 and no inclusion complex of delta-8 THC.

FIG. 7 is a bar graph illustrating the non-agitated and agitated dispersion efficiency for compositions with varying concentrations of effervescing agents.

### DETAILED DESCRIPTION

The present disclosure is based, in part, on the development of self-emulsifying cannabinoid compositions having enhanced dispersion properties, and methods of use thereof for producing cannabinoid inclusion complexes dispersed in a solution and for making a cannabinoid-infused food product or beverage. More particularly, the present disclosure relates to compositions and dosage forms of cannabinoids and cannabinoid related compounds, effervescing agents, and emulsifiers, which quickly self-emulsify to generate a uniform solution upon contact with liquid and produces a high concentration of dissolved cannabinoids, thereby possessing enhanced emulsification efficiency.

It has been unexpectedly discovered that it is possible to have a self-emulsifying effervescing system of cannabinoids, without the use of co-solvents, encapsulated oils, or extensive processing steps such as homogenization or high shear mixing, while reaching unprecedented concentrations of dispersed cannabinoids in solution. These systems and compositions have improved dispersibility and solution stability. For example, compositions described herein can comprise high dispersion efficiency for cannabinoids, reaching up to about 95% dispersion efficiency of added cannabinoid(s) within a couple of minutes exposure to an aqueous solution. Additionally, compositions systems and compositions described herein have improved emulsion stability as demonstrated by shelf-life testing, repeated centrifugation, and freeze/thaw cycling.

The present invention provides, in an aspect, a composition (Composition 1.0) comprising: (i) from about 20 to 95 wt % of an effervescing agent (ii) from about 5 to 80 wt % of a cannabinoid cyclodextrin inclusion complex, and (iii) from about 0 to 5 wt % of an emulsifier. For example, the invention includes:
1.1 Composition 1.0, wherein the amount of the effervescent agent in the composition is 25-95 wt %, 30-95 wt %, 35-95 wt %, 40-95 wt %, 45-95 wt %, 50-95 wt %, 55-95 wt %, 60-95 wt %, 65-95 wt %, 70-95 wt %, 75-95 wt %, 80-95 wt %, 85-95 wt %, 20-85 wt %, 25-85 wt %, 30-85 wt %, 35-85 wt %, 40-85 wt %, 45-85 wt %, 50-85 wt %, 55-85 wt %, 60-85 wt %, 65-85 wt %, 70-85 wt %, 75-85 wt %, or 80-85 wt %, optionally wherein the amount of the effervescent agent is 50-85 wt %.
1.2 Composition 1.1, wherein the amount of the effervescent agent in the composition is 65-85 wt %, optionally wherein the amount of the effervescent agent in the composition is 75-85 wt %.
1.3 Any of the preceding compositions, wherein the effervescent agent comprises an acidic agent and an alkaline agent.
1.4 Any of the preceding compositions, wherein the acidic agent is present in an amount of about 20 to about 50%, e.g., 40 to 50%, or 42 to 48%, by weight of the composition.
1.5 Any of the preceding compositions, wherein the acidic agent is selected from tartaric acid, citric acid, maleic acid, fumaric acid, malic acid, adipic acid, succinic acid, lactic acid, glycolic acid, alpha hydroxy acids, ascorbic acid, amino acids, their alkali metal acid salts, or combinations thereof.
1.6 Any of the preceding compositions, wherein the acidic agent is citric acid, ascorbic acid, or combinations thereof, optionally wherein the acidic agent is citric acid.
1.7 Any of the preceding compositions, wherein the alkaline agent is present in an amount of about 20 to about 50%, e.g., 30 to 40%, or 32 to 38%, by weight of the composition.
1.8 Any of the preceding compositions, wherein the alkaline agent is selected from carbonate, bicarbonate, sesquicarbonate and hydrogen carbonate salts of potassium, lithium, sodium, calcium, ammonium, or L-lysine carbonate, arginine carbonate, sodium glycine carbonate, sodium amino acid carbonate, or combinations thereof.
1.9 Any of the preceding compositions, wherein the alkaline agent is a sodium salt of bicarbonate, a potassium salt of bicarbonate, a sodium salt of carbonate, a potassium salt of carbonate or any combination thereof.
1.10 Any of the preceding compositions, wherein the alkaline agent is a combination of sodium bicarbonate and potassium carbonate.
1.11 Composition 1.10, wherein the composition comprises about 30 to about 40 wt % sodium bicarbonate and about 1 to about 10 wt % potassium carbonate, optionally wherein the composition comprises about 30 to about 36 wt % sodium bicarbonate and about 1 to about 6 wt % potassium carbonate, further optionally wherein the composition comprises about 30 to about 34 wt % sodium bicarbonate and about 1 to about 4 wt % potassium carbonate.
1.12 Any of the preceding compositions, wherein the relative acidic agent:alkaline agent ratio is from 10:1 to 1:10, optionally wherein the relative acidic agent:alkaline agent ratio is from 9:1 to 1:9, from 8:1 to 1:8. from 7:1 to 1:7, from 6:1 to 1:6, from 5:1 to 1:5, from 4:1 to 1:4, from 3:1 to 1:3, from 2:1 to 1:2, from 1.5:1 to 1:1.5, or from 1.3:1 to 1:1.3, further optionally wherein the relative acidic agent:alkaline agent ratio is from 1.3:1 to 1:1.3.
1.13 Any of the preceding compositions, wherein the amount of the cannabinoid cyclodextrin inclusion complex in the composition is 5 to 70%, 5 to 60%, 5-50 wt %, 5-40 wt %, 5-30 wt %, 5-20 wt %, 10-80 wt %, 10-70 wt %, 10-60 wt %, 10-50 wt %, 10-40 wt %, 10-30 wt %, 10-20 wt %, 10-15 wt %, or 15-20 wt %, optionally wherein the amount of the cannabinoid cyclodextrin inclusion complex in the composition is 10-20 wt %, further optionally wherein the amount of the cannabinoid cyclodextrin inclusion complex in the composition is 15-20 wt %.
1.14 Any of the preceding compositions, wherein the cannabinoid guest is delta-8 tetrahydrocannabinol (THC), delta-9 THC, delta-10 THC, cannabidiol (CBD), cannabinol (CBN), cannabigerol (CBG), hexahydrocannibinol (HHC), tetrahydrocannabivarin (THCV), tetrahydrocannabiphorol (THCP), 0-acetate thereof, isomers thereof, or combinations thereof, optionally wherein the cannabinoid guest is delta-8 THC or THC.
1.15 Any of the preceding compositions, wherein the cyclodextrin host is alpha-cyclodextrin, beta-cyclodextrin, gamma-cyclodextrin, 2-Hydroxypropyl-β-cyclodextrin, β-cyclodextrin sulfobutyl ether, or combinations thereof.
1.16 Any of the preceding compositions, wherein the cannabinoid cyclodextrin inclusion complex comprises about 5-25% cannabinoid and about 75-95% cyclodextrin by weight of the complex, optionally wherein the cannabinoid cyclodextrin inclusion complex comprises about 10-20 wt % cannabinoid and about 80-90 wt % cyclodextrin, further optionally wherein the cannabinoid cyclodextrin inclusion complex comprises about 15-20 wt % cannabinoid and about 80-85 wt % cyclodextrin.
1.17 Composition 1.16, wherein the cannabinoid cyclodextrin inclusion complex comprises about 16 wt % of cannabinoid and about 84 wt % cyclodextrin.
1.18 Any of the preceding compositions, wherein the amount of the emulsifier in the composition is 0-4 wt %, 0-3 wt %, 0-2 wt %, 0.05-5 wt %, 0.05-4 wt %, 0.05-3 wt %, 0.05-2 wt %, 0.5-5 wt %, 0.5-4 wt %, 0.5-3 wt %, or 0.5-2 wt %, optionally wherein the amount of the emulsifier in the composition is 0.5-5 wt %.
1.19 Any of the preceding compositions, wherein the emulsifier is a natural emulsifier, polysorbate 80, or a combination thereof, optionally wherein the emulsifier is polysorbate 80.
1.20 Any of the preceding compositions, wherein the composition further comprises one or more additives, optionally wherein the composition comprises about 5 to 50 wt %, e.g., 5 to 25 wt %, or 25 to 50 wt %, of the one or more additives.
1.21 Any of the preceding compositions, wherein the one or more additives is selected from the group consisting of terpenes, terpenoids, flavonoids, excipients, antioxidants, sweeteners, lubricants, colorants, vitamins, disintegrants, defoamers, foaming agents, wax, bitter blockers, and combinations thereof.
1.22 Any of the preceding compositions, wherein the composition has a dispersibility ranging from about 50 to 95%.
1.23 Any of the preceding compositions, wherein the composition has a shelf-life of at least about 45 days.
1.24 Any of the preceding compositions, wherein the composition has at least 2-times more dispersity than a control composition that does not comprise an emulsifier.
1.25 Any of the preceding compositions, wherein the composition is a powder, an effervescent powder, a tablet, an effervescent tablet, granules or effervescent granules.
1.26 Any of the preceding compositions, comprising
   (i) about 20 to about 50 wt % alkaline agent,
   (ii) about 20 to about 50 wt % acidic agent,
   (iii) about 5 to about 40 wt % cyclodextrin (e.g., β-cyclodextrin),
   (iv) about 1 to about 10 wt % cannabinoid, and
   (v) about 0 to about 10 wt %, e.g., 0.05 to 10 wt %, emulsifier.
1.27 Any of the preceding compositions, comprising
   (i) about 30 to about 40 wt % alkaline agent,
   (ii) about 40 to about 50 wt % acidic agent,
   (iii) about 10 to about 20 wt % cyclodextrin (e.g., β-cyclodextrin),
   (iv) about 1 to about 5 wt % cannabinoid, and
   (v) about 0 to about 5 wt % (e.g., 0.05 to 5 wt %) emulsifier.
1.28 Any of the preceding compositions, comprising
   (i) about 32 to about 38 wt % alkaline agent,
   (ii) about 42 to about 48 wt % acidic agent,
   (iii) about 12 to about 16 wt % cyclodextrin (e.g., β-cyclodextrin),
   (iv) about 1 to about 4 wt % cannabinoid, and
   (v) about 0 to about 4 wt % (e.g., 0.05 to 4 wt %) emulsifier.
1.29 Any of the preceding compositions, comprising
   (i) about 30 to about 40 wt % of a first alkaline agent,
   (ii) about 1 to about 10 wt % of a second alkaline agent,
   (iii) about 40 to about 50 wt % acidic agent,
   (iv) about 12 to about 20 wt % cyclodextrin (e.g., β-cyclodextrin),
   (v) about 1 to about 4 wt % cannabinoid, and
   (vi) about 0 to about 5 wt % (e.g., 0.05 to 5 wt %) emulsifier, optionally wherein the first alkaline agent is sodium bicarbonate and the second alkaline agent is potassium carbonate.
1.30 Any of the preceding compositions, comprising
   (i) about 30 to about 35 wt % of a first alkaline agent,
   (ii) about 1 to about 6 wt % of a second alkaline agent,
   (iii) about 42 to about 48 wt % acidic agent,
   (iv) about 12 to about 16 wt % cyclodextrin (e.g., β-cyclodextrin),
   (v) about 2 to about 4 wt % cannabinoid, and
   (vi) about 0 to about 4 wt % (e.g., 0.05 to 4 wt %) emulsifier, optionally wherein the first alkaline agent is sodium bicarbonate and the second alkaline agent is potassium carbonate.
1.31 Any of Compositions 1.0 to 1.25, comprising
   (i) about 20 to about 25 wt % sodium bicarbonate,
   (ii) about 1 to about 3 wt % potassium carbonate,
   (iii) about 30 to about 35 wt % citric acid,
   (iv) about 12 to about 20 wt % β-cyclodextrin,
   (v) about 5 to about 8 wt % cannabinoid (e.g., delta-8 THC or THC), and
   (vi) about 0-5 wt % Polysorbate 80.
1.32 Any of Compositions 1.0 to 1.25, comprising
   (i) about 30 to about 36 wt % sodium bicarbonate,
   (ii) about 1 to about 6 wt % potassium carbonate,
   (iii) about 40 to about 50 wt % citric acid,
   (iv) about 10 to about 18 wt % β-cyclodextrin,
   (v) about 1 to about 5 wt % cannabinoid (e.g., delta-8 THC or THC), and
   (vi) about 0 to 5 wt % (e.g., 0.05 to 5 wt %) Polysorbate 80.
1.33 Any of Compositions 1.0 to 1.25, comprising
   (i) about 30 to about 34 wt % sodium bicarbonate,
   (ii) about 1 to about 4 wt % potassium carbonate,
   (iii) about 42 to about 48 wt % citric acid,
   (iv) about 12 to about 16 wt % β-cyclodextrin,
   (v) about 1 to about 4 wt % cannabinoid (e.g., delta-8 THC or THC), and
   (vi) about 0 to 4 wt % (e.g., 0.05 to 4 wt %) Polysorbate 80.
1.34 Any of Compositions 1.0 to 1.25, comprising
   (i) about 32 wt % sodium bicarbonate,
   (ii) about 3 wt % potassium carbonate,
   (iii) about 45 wt % citric acid,
   (iv) about 14 wt % ß-cyclodextrin,
   (v) about 2.5 wt % cannabinoid (e.g., delta-8 THC or THC), and
   (vi) from about 0 to 2 wt % (e.g., 0.05 to 2 wt %) Polysorbate 80.

Effervescent Agents

Effervescent drug delivery preparations can be useful for quick production of solutions and for faster and better bioavailability of a drug or active ingredient. Additional benefits include eliminating the need to swallow large tablets or capsules, improved patient compliance, improved taste, more gentle action on a user's stomach, better dosing, appealing marketing aspects of fizzy substances, and general convenience of administration.

As used herein, “effervescence” means the evolution of bubbles of gas from a liquid as the result of a bubble or gas generating chemical reaction. The bubble or gas generating reaction of the effervescent coupled in the effervescent composition described herein can be the result of the reaction of an acidic agent and an alkaline agent. The reaction of these two general classes of compounds produces a gas upon contact with water or other aqueous solution. As used herein, an “effervescing agent” refers to the agent or agents that can be used in combination with an active ingredient of interest, such that upon contact with water or any other aqueous solution, an effervescence is generated (i.e., production of gas bubbles), and the active ingredient is dispersed in the water or aqueous solution.

In some aspects, an effervescing agent can comprise a combination of an acidic agent and an alkaline agent.

The term “acidic agent” refers to any compound or material that can serve as a proton source and can react with the alkaline agent to form a gas thereby causing a solution containing them to effervesce. An acidic agent can have more than one acid dissociation constant, i.e., more than one acid functional group. In some embodiments, the acidic agent is present in an amount of about 20 to about 50%, e.g., 40 to 50%, or 42 to 48%, by weight of the composition. An acidic agent can be any organic or inorganic acid in the free acid, acid anhydride and acid salt form. An acidic agent that is in solid state at room temperature and shows pH 4.5 or lower when saturated into water at room temperature or its acid alkali metal salts (e.g., sodium salt, potassium salt, etc.) can be employed. As the acidic agent for the effervescent compositions described herein, a compound that is not harmful to animals including man is desirably employed. Non-limiting examples of acidic agents include tartaric acid, citric acid, maleic acid, fumaric acid, malic acid, adipic acid, succinic acid, lactic acid, glycolic acid, alpha hydroxy acids, ascorbic acid, amino acids and their alkali metal acid salts. In various aspects, an acidic agent can be citric acid, ascorbic acid, or a combination thereof. In some embodiments, the acidic agent is citric acid. An acidic agent can be in a liquid state at room temperature, e.g., phosphoric acid or pyrophosphoric acid, while their acid alkali metal salts are solid at room temperature. in such cases, the acid alkali metal salts can be employed as acidic agents. In an aspect acidic agents can have a relatively large acid dissociation constant (Ka) (e.g., about 100, 101, 102, 103 or more) and a small hygroscopicity (critical humidity at 30° C. is 40% RH or more). In some aspects, an acidic agent can dissolve rapidly (if not essentially instantaneously) in water or an aqueous solution. For example, an acidic agent can dissolve in about 60, 30, 20, 10, 5, 2, 1, or fewer seconds.

The term “alkaline agent” means an alkaline compound that releases a gas, or causes a solution to effervesce, when exposed to a proton source such as an acidic agent or water. The alkaline agent can be a carbon dioxide gas precursor. In some embodiments, the alkaline agent is present in an amount of about 20 to about 50%, e.g., 30 to 40%, or 32 to 38%, by weight of the composition.

When the alkaline agent is a carbon dioxide precursor, compounds such as carbonate, bicarbonate, sesquicarbonate and hydrogen carbonate salts (herein, carbonate and hydrogen carbonate, or bicarbonate, are generically referred to as carbonate) of potassium, lithium, sodium, calcium, ammonium, or L-lysine carbonate, arginine carbonate, sodium glycine carbonate, sodium amino acid carbonate can be used.

In various aspects, the alkaline agent can be a sodium salt of bicarbonate, a potassium salt of bicarbonate, a sodium salt of carbonate, a potassium salt of carbonate or any combination thereof. In some embodiments, the alkaline agent is a combination of sodium bicarbonate and potassium carbonate. In some embodiments, the composition comprises about 30 to about 36 wt % sodium bicarbonate and about 1 to about 6 wt % potassium carbonate. In some embodiments, the composition comprises about 30 to about 34 wt % sodium bicarbonate and about 1 to about 4 wt % potassium carbonate. In some embodiment, the composition comprises about 20 to about 25 wt % sodium bicarbonate and about 1 to about 3 wt % potassium carbonate.

Where the effervescent agent includes two mutually reactive components, such as an acidic agent and an alkaline agent both components can react completely. A ratio of components that provides for equal amounts of reaction equivalents can be used to ensure the components react completely. For example, if the acid used is diprotic, then either twice the amount of a mono-reactive carbonate alkaline agent, or an equal amount of an all-reactive alkaline agent should be used for complete neutralization to be realized. However, in other embodiments, the amount of either the acidic agent or the alkaline agent can exceed the amount of the other component such that the components do not react completely. This can be useful to enhance taste and/or performance of an effervescent composition described herein containing an average of either component. By controlling the relative ratio of acidic agent:alkaline agent, the effervescent composition can be used to regulate the pH of the environment. In some embodiments, the relative acidic agent:alkaline agent ratio is from 10:1 to 1:10, e.g., from 9:1 to 1:9, from 8:1 to 1:8. from 7:1 to 1:7, from 6:1 to 1:6, from 5:1 to 1:5, from 4:1 to 1:4, from 3:1 to 1:3, from 2:1 to 1:2, from 1.5:1 to 1:1.5, or from 1.3:1 to 1:1.3. For example, the relative acidic agent:alkaline agent ratio can be 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10.

The ratio of an acidic agent to an alkaline agent can also be determined according to the pH required for dissolving an additional ingredient(s) included in an effervescent composition. When the solubility of the additional ingredient(s) increases at the acid side, the pH of the solution is lowered by adding the acidic agent in an amount more than equivalent to the alkaline agent. When the solubility of the additional ingredient(s) increases at the basic side, the pH of the solution is raised by adding the alkaline agent in an amount more than equivalent to the acidic agent. In either case, the pH near the acidic agent immediately after the dissolution is low, while the pH near an alkaline agent is high. In a case where the solubility of the additional ingredient(s) does not depend on pH, the ratio of an acidic agent and an alkaline agent can be optionally selected.

An amount of an alkaline agent such as a carbon dioxide precursor, to be incorporated can be proportional to the volume of gas, e.g., carbon dioxide gas, generated. When it is desired to increase the dissolution rate of an additional ingredient included in an effervescent composition described herein, it can be advantageous to increase the amount of an alkaline agent such as carbon dioxide precursor accordingly.

An acidic agent and an alkaline agent such as a carbon dioxide precursor can be used in a powdery or granular state, usually 90% or more of the granulates being capable of passing through a mesh screen. A mesh screen can have pore having a size ranging from about 50 μm to 1000 μm. For example, a mesh screen can have a pore size of about 50 μm, 100 μm, 150 μm, 200 μm, 250 μm, 500 μm, 750 μm, 1000 μm or more.

Salt saturation can be used to maintain particular values of relative humidity, and to protect from humidity. To prevent premature effervescence of the compositions described herein, an alkaline agent can be modified to be more resistant to humidity. Such modification can include saturation of the alkaline agent with a salt. In certain embodiments the alkaline agent, e.g., bicarbonate can be surface modified with a carbonate salt for the purpose of increasing resistance to humidity and premature effervescence.

In some embodiment, the amount of effervescent agent in the compositions as described herein is about 20 to 95 wt %. For example, the amount of effervescent agent in the composition can be about 20-95 wt %, 25-95 wt %, 30-95 wt %, 35-95 wt %, 40-95 wt %, 45-95 wt %, 50-95 wt %, 55-95 wt %, 60-95 wt %, 65-95 wt %, 70-95 wt %, 75-95 wt %, 80-95 wt %, 85-95 wt %, 20-85 wt %, 25-85 wt %, 30-85 wt %, 35-85 wt %, 40-85 wt %, 45-85 wt %, 50-85 wt %, 55-85 wt %, 60-85 wt %, 65-85 wt %, 70-85 wt %, 75-85 wt %, or 80-85 wt %. In one embodiment, the amount of effervescent agent in the compositions describes herein in about 65-85 wt %.

Cyclodextrin Inclusion Complexes

Compositions described herein can comprise a cannabinoid cyclodextrin inclusion complex. In some embodiments, the amount of the cannabinoid cyclodextrin inclusion complex in the composition is 5 to 80%, e.g., 5 to 70%, 5 to 60%, 5-50 wt %, 5-40 wt %, 5-30 wt %, 5-20 wt %, 10-80 wt %, 10-70 wt %, 10-60 wt %, 10-50 wt %, 10-40 wt %, 10-30 wt %, 10-20 wt %, 10-15 wt %, or 15-20 wt %. Cyclodextrins are a family of cyclic oligosaccharides, which are comprised of a macrocyclic ring of glucose subunits joined by α-1,4 glycosidic bonds. Cyclodextrins are produced from starch by enzymatic conversion. As used herein, a “cyclodextrin inclusion complex” is a complex based on noncovalent associations between a cyclodextrin host and a guest. In this case, the guest is one or more cannabinoids. Noncovalent associations can improve water solubility, bioavailability, and stability of the guest molecule. Guest molecules can be released from the complexes upon ingestion by a consumer.

Cyclodextrins are composed of 5 or more α-D-glucopyranoside units linked 1->4, as in amylose (a fragment of starch). The largest cyclodextrin contains 32 1,4-anhydroglucopyranoside units, while as a poorly characterized mixture, at least 150-membered cyclic oligosaccharides are also known. Typical cyclodextrins contain a number of glucose monomers ranging from six to eight units in a ring, creating a cone shape:
α (alpha)-cyclodextrin: 6 glucose subunits;
β (beta)-cyclodextrin: 7 glucose subunits;
γ (gamma)-cyclodextrin: 8 glucose subunits.

Cyclodextrins have a hydrophobic interior and hydrophilic exterior, which can form complexes with hydrophobic compounds. Alpha-, beta-, and gamma-cyclodextrin are all generally recognized as safe by the U.S. FDA. A cyclodextrin confers solubility and stability to the guest.

Cyclodextrin molecule includes primary and secondary hydroxyl groups and includes a hydrophilic outer portion. The cyclodextrin molecule also contains carbon atoms, hydrogen atoms, and ether bonds, and includes a hydrophobic three-dimensional indentation. The hydrophobic dimple of the cyclodextrin molecule can act as a host and hold various molecules containing the hydrophobic moiety, or the guest, to form a cyclodextrin inclusion complex.

As used herein, the term “inclusion complex” refers to any cannabinoid guest that can be retained or trapped within a three-dimensional cavity that at least a portion of which is present in a cyclodextrin molecule. In various aspects, the cannabinoid cyclodextrin inclusion complex can comprise a cyclodextrin host and a cannabinoid guest. In some embodiments, the cannabinoid cyclodextrin inclusion complex comprises about 5-25 wt % cannabinoid and about 75-95 wt % cyclodextrin. In some embodiments, the cannabinoid cyclodextrin inclusion complex comprises about 10-20 wt % cannabinoid and about 80-90 wt % cyclodextrin. In some embodiments, the cannabinoid cyclodextrin inclusion complex comprises about 15-20 wt % cannabinoid and about 80-85 wt % cyclodextrin.

Encapsulation

In some embodiments, the compositions described herein can be formulated as spray dry composition, and not include cyclodextrin as a host for the cannabinoid compound.

In an embodiment, one or more cannabinoids can be encapsulated or microencapsulated, which are a techniques where in a bioactive compound is encapsulated by a biopolymer, protecting it from oxygen, water or other conditions to improve its stability. Encapsulation and microencapsulation promotes easier handling and prevents lumping, improving flowability, compression and mixing properties, reducing core particle dustiness, and modifying particle density. It also enhances the shelf life of the encapsulated compound. Non-limiting examples of encapsulation and microencapsulation technologies include spray drying and freeze drying. Spray drying processes includes, for example, the dispersion of the core material in an entrapment material (e.g., an encapsulating agent), followed by atomization and spraying of the mixture in a hot air desiccant into a chamber. A spray dryer can generally be operated at inlet temperature ranging from about 90 to 120° C. Dry liposomal formulations of one or more cannabinoids can be freeze dried to encapsulate the cannabinoids. Cannabinoids can be formulated with any suitable liposome system and freeze dried.

Non limiting examples of encapsulating agents that can be used in encapsulation of the compositions described herein include polysaccharides (such as starches, maltodextrins corn syrups, sugars, guar gum, xanthan gum, Arabic gum and other gums), lipids, proteins (such as gelatin, casein, soy and wheat protein), glycoproteins and pectin.

Cannabinoids

Cannabis is a genus of flowering plants in the family Cannabaceae, the term “hemp” is more often used to refer only to varieties of Cannabis cultivated for non-drug use. Cannabis has long been used for hemp fiber, hemp seeds and their oils, hemp leaves for use as vegetables and as juice, medicinal purposes, and as a recreational drug. Cannabis plants produce a group of chemicals called cannabinoids, which produce mental and physical effects when consumed, and which have increased their study and use for medicinal purposes. As used herein, the terms “cannabis” and “cannabinoid” can be used interchangeably. Cannabis and cannabinoids can be present as a cyclodextrin inclusion complex guest as the active ingredient of compositions described herein.

Cannabis compositions developed for ingestion can overcome the health risks and social challenges associated with smoking and vaporization methods. Cannabinoid edibles make up a significant proportion of total cannabinoid use globally, however, a considerable percentage of edible cannabinoid products are not adapted for use in casual recreational and social settings, or routine retail sales to consumers.

Cannabinoids, terpenoids, and other compounds are secreted by glandular trichomes that occur most abundantly on the floral calyxes and bracts of female plants. A cannabinoid is one of a class of diverse chemical compounds that acts on cannabinoid receptors, which are part of the endocannabinoid system found in cells that alter neurotransmitter release in the brain. Ligands for these receptor proteins include the endocannabinoids produced naturally in the body by animals; phytocannabinoids, found in cannabis; and synthetic cannabinoids, manufactured artificially.

At least 113 different cannabinoids have been isolated from the Cannabis plant. The most notable and best studied cannabinoids include the phytocannabinoid tetrahydrocannabinol (THC), the primary psychoactive compound in cannabis; cannabidiol (CBD) and cannabinol (CBN).

As used herein the term “cannabinoids” refers to cannabinoids derived from cannabis plants and synthetic cannabinoids and synthetic cannabinoid related compounds and includes, but is not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), A6-Cannabidiol (A6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or A9-THC), A8-tetrahydrocannabinol (D8-THC), frans-DI O-tetrahydrocannabinol (frans-AIO-THC), c/s-DI O-tetrahydrocannabinol (c/s-A10-THC), Tetrahydrocannabinolic acid C4 (THCA-C4), Tetrahydrocannbinol C4 (THC C4), Tetrahydrocannabivarinic acid (THCVA), Tetrahydrocannabivarin (THCV), A8-Tetrahydrocannabivarin (Dd-THCV), A9-Tetrahydrocannabivarin (A9-THCV), Tetrahydrocannabiorcolic acid (THCA-C1), Tetrahydrocannabiorcol (THC-C1), A7-c/s-iso-tetrahydrocannabivarin, A8-tetrahydrocannabinolic acid (Dd-THCA), A9-tetrahydrocannabinolic acid (A9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-A9-tetrahydrocannabinol (11-OH-THC), 1 1-nor-9-carboxy-A9-tetrahydrocannabinol, Ethoxy-cannabitriolvarin (CBTVE), 10-Ethoxy-9-hydroxy-A6a-tetrahydrocannabinol, Cannabitriolvarin (CBTV), 8,9-Dihydroxy-A6a(10a)-tetrahydrocannabinol (8,9-Di-OH-CBT-C5), Dehydrocannabifuran (DCBF), Cannbifuran (CBF), Cannabichromanon (CBCN), Cannabicitran (CBT), 10-Oxo-A6a(10a)-tetrahydrocannabinol (OTHC), A9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol (HHC), beta-hydroyx one, and Dodeca-2E,4E-dienoic acid isobutylamide, 0-acetate esters, or any combination thereof.

Tetrahydrocannabinol (THC) is the primary psychoactive component of the Cannabis plant, found as delta-9-tetrahydrocannabinol (A9-THC, THC) and delta-8-tetrahydrocannabinol (A8-THC); which produce the effects associated with cannabis by binding to the CB1 cannabinoid receptors in the brain. There is some scientific evidence supporting the effectiveness of the cannabis extracts in treating certain symptoms of multiple sclerosis and pain, especially centrally mediated pain and painful spasms. The effects of THC on various symptoms observed in neurodegenerative diseases and disorders, such as Huntington disease, Parkinson's disease, Alzheimer's disease or Tourette syndrome have been assessed in trials.

Cannabidiol (CBD) is a phytocannabinoid discovered in 1940. It accounts for up to 40% of the cannabis plant cannabinoid's extract. Clinical research on cannabidiol includes preliminary studies of anxiety, cognition, movement disorders, and pain. Cannabidiol can be taken into the body in multiple ways, including by inhalation of cannabis smoke or vapor, as an aerosol spray into the cheek, and by mouth. It can be supplied as CBD oil containing only CBD as the active ingredient (no included tetrahydrocannabinol [THC] or terpenes), a full-plant CBD-dominant hemp extract oil, capsules, dried cannabis, or as a prescription liquid solution. CBD does not have the same psychoactivity as THC and can change the effects of THC on the body if both are present.

Cannabigerol (CBG) is the non-acidic form of cannabigerolic acid, the parent molecule from which other cannabinoids are synthesized.

During growth of the plant, most of the cannabigerol is converted into other cannabinoids, primarily tetrahydrocannabinol (THC) or cannabidiol (CBD), leaving about 1% cannabigerol in the plant, therefore, CBG is considered a minor constituent of cannabis. Contrary to the major psychoactive cannabinoid THC, CBG is non-psychoactive but still contributes to the overall effects of Cannabis.

In one aspect, a cannabinoid guest can be delta-8 tetrahydrocannabinol (THC), delta-9 THC, delta-10 THC, cannabidiol (CBD), cannabinol (CBN), cannabigerol (CBG), hexahydrocannibinol (HHC), tetrahydrocannabivarin (THCV), tetrahydrocannabiphorol (THCP), 0-acetate thereof, or combinations thereof.

In some aspects, a cyclodextrin host can be alpha-cyclodextrin, beta-cyclodextrin, gamma-cyclodextrin, 2-Hydroxypropyl-β-cyclodextrin, β-cyclodextrin sulfobutyl ether, or combinations thereof.

In various aspects, a cannabinoid cyclodextrin inclusion complex can comprise a cyclodextrin host and a cannabinoid guest. For example, a cannabinoid cyclodextrin inclusion complex can comprise: an alpha-cyclodextrin and a THC guest, a CBD guest, a CBG guest, an HHC guest, a THCV guest, or a THCP guest; a beta-cyclodextrin and a THC guest, a CBD guest, a CBG guest, an HHC guest, a THCV guest, or a THCP guest; or a gamma-cyclodextrin and a THC guest, a CBD guest, a CBG guest, an HHC guest, a THCV guest, or a THCP guest.

In an aspect, a cannabinoid cyclodextrin inclusion complex can comprise about 5, 10, 15, 16, 20 or more wt % of cannabinoid and about 75, 80, 84, 85, 90, 95 or more wt % cyclodextrin. In various aspects, a cannabinoid cyclodextrin inclusion complex can comprise about 16 wt % of cannabinoid and about 84 wt % cyclodextrin.

Cannabinoid cyclodextrin inclusion complexes can be prepared via any suitable method including, for example, through a kneading method, co-precipitation method, or ethanolic solution method (described by Semcheddine et al., AAPS Pharm Sci Tech. 2015; 16(3):704-715, or by Mannila et al., J. Pharmaceutical Sciences 2007; 96(2)313:319, or by Miclea et al., Farmacia 2010; 58(5)583-593, for example).

The formulations described herein can be suitable for drug compounds other than cannabinoids. In some embodiments, the compositions described herein comprise an oily drug that is not a cannabinoid as a guest for the cyclodextrin inclusion complex.

Emulsifiers

In some aspects, compositions can further comprise an emulsifier. As used herein, the term “emulsifier” refers to an additive which helps two liquids mix. An emulsifier comprises of a water-loving hydrophilic head and an oil-loving hydrophobic tail. The hydrophilic head is directed to the aqueous phase and the hydrophobic tail to the oil phase. The emulsifier positions itself at the oil/water or air/water interface and, by reducing the surface tension, has a stabilizing effect on the emulsion.

In some embodiments, the composition comprises from about 0% to about 5% (e.g., about 0, 0.01, 0.1, 1, 1.5, 2, 2.5, 3, 3.5, 4, 4.5, or 5%) by weight of an emulsifier. In some embodiments, the amount of the emulsifier is 0-4 wt %, 0-3 wt %, 0-2 wt %, 0.05-5 wt %, 0.05-4 wt %, 0.05-3 wt %, 0.05-2 wt %, 0.5-5 wt %, 0.5-4 wt %, 0.5-3 wt %, or 0.5-2 wt %.

In some aspects, the emulsifier can be a surfactant. As used herein the term “surfactant” refers to a compound that reduces surface tension when dissolved in water. The ingredients in the compositions described herein have varying consistencies, viscosities, and specific gravities, and therefore also have varying levels of surface tension. That surface tension, along with the chemical makeup of the ingredients, can prevent optimal mixing and/or dispersion in liquids. Separation naturally results in the heavier substance sinking, with the lighter floating on top. The addition of a surfactant can help reducing that surface tension, acting as an emulsifier, allowing the ingredients to more easily homogenize without the physical pressure to separate. Surfactants ensure that finished products and ingredients hold their emulsion during transport and storage. Surfactants are a very large category that include several specific types, many of which are naturally derived. For example, lecithin occurs naturally in both plant and animal tissues, including eggs, and is widely regarded as one of the most common emulsifiers. Similarly, fatty acids and certain types of cholesterol are effective surfactants.

In some aspects, the surfactant can be a natural emulsifier or polysorbate 80. Non-limiting examples of natural surfactants include alkyl glycoside, carrageenan (carbohydrate), cholesterol, lanolin, lecithin, monoglyceride (fatty acid), phytosterol, protein, emulsion salts (such as sodium hexametaphosphate, sodium citrate, and potassium salts), and tea saponin extract. Non-limiting examples of emulsifier include polysorbate 80, oleoyl polyoxyl-6 glycerides, polyoxyl 35 hydrogenated castor oil, sucrose distearate, tocopherol polyethylene glycol 1000 succinate, lauroyl polyoxyl-32 glycerides, sorbitan monooleate, salts thereof, derivatives thereof, and mixtures of emulsifiers.

Additives

In some aspects, compositions can further comprise one or more additives. As used herein, the term “additive” refers to any agent that can be added to the compositions described herein to, e.g., provide advantageous formulation properties. The one or more additives can be, e.g., terpenes, terpenoids, flavonoids, excipients, antioxidants, sweeteners, lubricants, colorants, vitamins, disintegrants, defoamers, foaming agents, wax, bitter blocking agents, any other suitable additive, and combinations thereof.

In some embodiments, the composition comprises about 5% to about 50% by weight of an additive or a mixture of additives. In certain embodiments, the composition comprises about 5% to about 25%, e.g., 5% to 10%, 10% to 15%, 15% to 20%, 20% to 25% (e.g., about 5, 10, 15, 20, 25%) by weight of an additive or a mixture of additives. In certain embodiments, the composition can comprise about 25% to about 50%, e.g., 25% to 30%, 30% to 35%, 35% to 40%, 40% to 45%, 45% to 50% (e.g., about 25, 30, 35, 40, 45, 50%) by weight of an additive or a mixture of additives.

In one aspect, a composition can comprise one or more terpenes. The term “terpene” as used herein includes terpenoids. Terpenes are lipophilic compounds, volatile and liquid at room temperature and can be used in the compositions described herein as solubilizing agents. Terpenes are major secondary metabolites of cannabis and are responsible for the odor and flavor of various cannabis strains. Cannabis strains and hemp strains produce many terpenes as secondary metabolites. Terpenes are synthesized from terpene unit into monoterpenes, sesquiterpenes, di-terpenes that are lipophilic, volatile and insoluble in water and are cyclic or bicyclic or not cyclic and can have alcohol, aldehyde or ketone chemical moiety. The term “terpene” also includes essential oils. As used herein, the term “essential oil” refers to concentrated plant extracts. Essential oils can have anti-bacterial, antiviral, antifungal properties, and can provide mood boost, stress reduction, increased attentiveness, improved sleep, anxiety and pain reduction, inflammation reduction, and nausea and headaches relieve. Non-limiting examples of essential oils include: lavender oil, tea tree oil, frankincense oil, peppermint oil, eucalyptus oil, lemon oil, lemongrass oil, orange oil, rosemary oil, bergamot oil and cedarwood oil. The term “terpene” does not include fats and/or lipids.

In certain embodiments, compositions described herein comprise about 0-2% by weight of a terpene or a mixture of terpenes. In certain embodiments, the composition comprises about 0.01% to about 5% (e.g., about 0.01, 0.1, 0.2, 0.5, 1, 2, 3, 4, or 5%) by weight of a terpene or a mixture of terpenes. Terpenes can have different flavors, which may impact the amount of terpenes one may want to use in a composition for consumption. One of skill in the art would readily be able to evaluate which terpenes to use in lower amount (e.g., those with the strongest or less desirable flavors), and which terpenes to use in higher amount (e.g., those with the lightest or more favorable flavors). In certain embodiments, the terpene is selected from the group consisting of bisabolol, borneol, caryophyllene, carene, camphene, cineol, citronella, eucalyptol, geraniol, guaiol, humulene, isopropyltoluene, isopulegol, linalool, limonene, menthol, myrcene, nerolidol, ocimene, pinene, phytol, pulegone, terpinene, terpinolene, thymol, salts thereof, derivatives thereof, and mixtures of terpenes.

In another aspect, a composition can comprise one or more flavonoids. The term “flavonoid” as used herein refers to a class of polyphenolic secondary metabolites found in plants, and thus commonly consumed in the diets of humans. Chemically, flavonoids have the general structure of a 15-carbon skeleton, which consists of two phenyl rings (A and B) and a heterocyclic ring (C, the ring containing the embedded oxygen). This carbon structure can be abbreviated C6-C3-C6. According to the IUPAC nomenclature, they can be classified into flavonoids or bioflavonoids, isoflavonoids, derived from 3-phenylchromen-4-one (3-phenyl-1,4-benzopyrone) structure, and neoflavonoids, derived from 4-phenylcoumarine (4-phenyl-1,2-benzopyrone) structure. The three flavonoid classes are all ketone-containing compounds and as such, anthoxanthins (flavones and flavonols). This class was the first to be termed bioflavonoids.

In one aspect, a composition can comprise one or more excipients. An “excipient” as used herein is a substance formulated alongside an active ingredient of a composition included for the purpose of long-term stabilization, bulking up solid formulations that contain potent active ingredients in small amounts (thus often referred to as “bulking agents”, “fillers”, or “diluents”), or to confer an enhancement on the active ingredient in the final dosage form, such as facilitating its absorption, reducing its viscosity, or enhancing its solubility. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life.

In another aspect, a composition can comprise one or more sweeteners. As used herein, the term “sweetener” refers to agents that can be added to a composition to make the ingredients more palatable, especially in chewable tablets. Sugar can be used to mask unpleasant tastes or smells, but artificial sweeteners tend to be preferred, as natural ones tend to cause tooth decay. A sugar substitute is a food additive that provides a sweet taste like that of sugar while containing significantly less food energy than sugar-based sweeteners, making it a zero-calorie (non-nutritive) or low-calorie sweetener. Artificial sweeteners can be derived through manufacturing of plant extracts or processed by chemical synthesis. Sugar substitute products are commercially available in various forms, such as small pills, powders, and packets. Non-limiting examples of sugar substitutes include aspartame, monk fruit extract, saccharin, sucralose, cyclamate, acesulfame K, neotame and stevia. Additionally, sugar alcohols such as erythritol, xylitol, and sorbitol can be used as sweeteners.

In one aspect, a composition can comprise one or more lubricants. The term “lubricant” as used herein refers to any agent that can be used to prevent ingredients from clumping together and from sticking to tablet punches, capsule filling machines, or other machinery. Lubricants also ensure that tablet formation and ejection can occur with low friction between the solid and die wall. Common minerals like talc or silica, and fats, e.g., vegetable stearin, magnesium stearate or stearic acid are the most frequently used lubricants. Other lubricants for effervescent tablets can include polyethylene glycol or sodium stearyl fumarate. While lubricants can be added to improve manufacturability of drug products, it can also negatively impact the product quality. For example, extended mixing of lubricants during blending can result in delayed dissolution and softer tablets, which is often referred to as “over-lubrication”. Therefore, optimizing lubrication time can be important in formulation of compositions.

In another aspect, a composition can comprise one or more colorants. As used herein, the term “colorant” refers to an agent that can add color to a composition, which can be used to improve the appearance of a formulation. Color consistency is important as it allows easy identification of a composition. Furthermore, colors often improve the aesthetic look and feel of a composition. Small amounts of coloring agents are easily processed by the body. Coloring agents include titanium dioxide, iron oxides such as petal or iron yellow, and pigments suitable for food such as pigments known as FD & C pigments, and grape skin extract, red beet powder, beta-carotene, annatto. Natural colorants such as carmine, turmeric, and paprika can be included. Coloring agents can be used in conventional amounts, for example, in the range of about 0.001 to about 1 percent by weight of the entire dosage form.

In one aspect, a composition can comprise one or more vitamins, or other nutraceutical agents. As used herein, the term “vitamin” refers to any organic molecule that is an essential micronutrient which an organism needs in small quantities for the proper functioning of its metabolism. Essential nutrients cannot be synthesized in the organism, either at all or not in sufficient quantities, and therefore must be obtained through the diet. Non-limiting examples of vitamin include: vitamin A (as all-trans-retinol, all-trans-retinyl-esters, as well as all-trans-beta-carotene and other provitamin A carotenoids), vitamin 1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (phylloquinone and menaquinones). As used herein, the term “nutraceutical” refers to a pharmaceutical alternative which claims physiological benefits, including dietary supplements and food additives. Non-limiting examples of nutraceutical include melatonin.

In another aspect, a composition can comprise a bitter blocking. As used herein, the term “bitter blocker” or “masking agent” refers to bitter blocking flavors that reduce the taste of bitterants. The most common bitterants are caffeine, plant-based proteins, theobromine (found in high cacao dark chocolate), phenylindanes (chlorogenic acid found in dark roast coffee), cannabinoids, low purity rebaudioside A, OTC active pharma ingredients (dextromethorphan, guaifenesin, and omeprazole), quinine, vitamin and minerals, grains, and many other functional ingredients. Non-limiting examples of bitter blockers can include salts such as potassium salt, sodium salts and sodium chloride, adenosine monophosphate, sodium acetate, sodium gluconate, monosodium glutamate, adenosine 5′monophosphate, neohesperidin dihydrochalcone, thaumatin (E957), derivatives of cinnamic acid, miraculin, and lipoproteins.

In one aspect, a composition can comprise one or more preservatives. The compositions described herein can include “preservatives”, which, as used herein can include vitamins and/or antioxidants. Some typical preservatives used in pharmaceutical formulations are antioxidants like vitamin A, vitamin E, vitamin C, retinyl palmitate, and selenium; the amino acids cysteine and methionine; citric acid and sodium citrate. Synthetic preservatives can include parabens such as methyl paraben and propyl paraben.

In another aspect, a composition can comprise one or more disintegrants. “Disintegrants” can be used in the compositions describes herein as they expand and dissolve when wet causing a solid composition (such as a tablet or granule for example) to break apart, releasing the active ingredients for optimal absorption. They ensure that when the composition is in contact with water, it rapidly breaks down into smaller fragments, facilitating dissolution. Non-limiting examples of disintegrants include crosslinked polymers (e.g., crosslinked polyvinylpyrrolidone, crospovidone, crosslinked sodium carboxymethyl cellulose, croscarmellose sodium) and the modified starch sodium starch glycolate.

In one aspect, a composition can comprise one or more flavors. “Flavors” can be used to mask unpleasant tasting active ingredients and improve the acceptance of a composition. Flavorings can be natural (e.g., fruit extract) or artificial. For example, to improve (i) a bitter product, mint, cherry or anise can be used, (ii) a salty product, peach, apricot or licorice can be used, (iii) a sour product, raspberry or licorice can be used, (iv) an excessively sweet product, vanilla can be used.

In another aspect, a composition can comprise one or more defoamers. A “defoamer” or “an anti-foaming” agent as used herein is a chemical additive that reduces and hinders the formation of foam. The terms anti-foam agent and defoamer are often used interchangeably. Strictly speaking, defoamers eliminate existing foam and anti-foamer prevent the formation of further foam. Commonly used agents are insoluble oils, polydimethylsiloxanes and other silicones, certain alcohols, stearates and glycols. The additive can be used to prevent formation of foam or can be added to break a foam already formed.

In one aspect, a composition can comprise one or more foaming agents. A “foaming agent” is a material that facilitates the formation of foam such as a surfactant or a blowing agent. A surfactant, when present in small amounts, reduces surface tension of a liquid (reduces the work needed to create the foam) or increases its colloidal stability by inhibiting coalescence of bubbles. A blowing agent is a gas that forms the gaseous part of the foam.

In another aspect, a composition can comprise one or more waxes. “Waxes” are a diverse class of organic compounds that are lipophilic, malleable solids near ambient temperatures. They include higher alkanes and lipids, typically with melting points above about 40° C. (104° F.), melting to give low viscosity liquids. Waxes are insoluble in water but soluble in organic, nonpolar solvents. Natural waxes of different types are produced by plants and animals and occur in petroleum.

Dispersibility

In some aspects, compositions can have a dispersibility ranging from about 50 to 95% (e.g., about 50, 60, 70, 80, 90, 95% or more).

Dispersion is a process by which (in the case of solid dispersing in a liquid) agglomerated particles are separated from each other, and a new interface between the inner surface of the liquid dispersion medium and the surface of the dispersed particles is generated. This process is facilitated by molecular diffusion and convection. With respect to molecular diffusion, dispersion occurs as a result of an unequal concentration of the introduced material throughout the bulk medium. When the dispersed material is first introduced into the bulk medium, the region at which it is introduced then has a higher concentration of that material than any other point in the bulk. This unequal distribution results in a concentration gradient that drives the dispersion of particles in the medium so that the concentration is constant across the entire bulk. With respect to convection, variations in velocity between flow paths in the bulk facilitate the distribution of the dispersed material into the medium.

Although both transport phenomena contribute to the dispersion of a material into the bulk, the mechanism of dispersion is primarily driven by convection in cases where there is significant turbulent flow in the bulk. Diffusion is the dominant mechanism in the process of dispersion in cases of little to no turbulence in the bulk, where molecular diffusion is able to facilitate dispersion over a long period of time. Stirring the mixture can create turbulent flows in the liquid and accelerate the process of dispersion through convection-dominated dispersion.

The term dispersion also refers to the physical property of the degree to which particles clump together into agglomerates or aggregates. A full quantification of dispersion can involve the size, shape, and number of particles in each agglomerate or aggregate (e.g., cannabinoid inclusion complexes), the strength of the interparticle forces, their overall structure, and their distribution within the system. However, the complexity is usually reduced by comparing the measured size distribution of “primary” particles to that of the agglomerates or aggregates. When discussing suspensions of solid particles in liquid media, the zeta potential is most often used to quantify the degree of dispersion, with suspensions possessing a high absolute value of zeta potential being considered as well-dispersed.

As used herein, the dispersion of the cannabinoid inclusion complexes can be referred to as a percent dispersion, which reflect the percent of cannabinoid inclusion complexes that is dispersed, as opposed to the remaining cannabinoid inclusion complexes that are not dispersed (remaining in a solid state). A percent dispersion can range from 0%, when none of the cannabinoid inclusion complexes are dispersed into the medium, to 100% when virtually all the cannabinoid inclusion complexes are dispersed in the medium. the composition described herein can have a dispersibility ranging from about 50 to 95%. For example, the dispersibility of the composition can be 50%, 55%, 60%, 65%, 70%, 75%, 80%, 85%, 90%, 95% or more.

The compositions described herein can retain at least 2 times the concentration of cannabinoid in solution (e.g., about at least 2, 2.5, 3, 3.5, 4.0 times or more). By “retaining concentration of a cannabinoid” in solution, it is meant that the compositions can have better or improved dispersion efficiency (DE). DE can be measured as the amount of a compound in solution reported to the amount of the compound introduced in the solution:

Dispersion efficiency (DE)=(Measured amount of cannabinoid in solution)/(Amount of cannabinoid introduced to solution)

Shelf-Life

As used herein, the term “shelf-life” is the length of time that the compositions described herein can be stored without becoming unfit for use, consumption, or sale. By “shelf” it is meant that the storage conditions include room temperature (e.g., from about 15 to 30° C., such as about 15, 16, 17, 18, 19, 20, 21, 22, 23, 24, 25, 26, 27, 28, 29 or 30° C.), as well as minimal/reduced exposure to direct sunlight. For example, the compositions described herein can have a shelf-life of at least 15, 20, 30, 40, 45, 50, 55, 60, 65, 70, 75, 80, 85, 90, 180, 365, or more days. A shelf-life can mean that the composition retains its dispersibility efficiency over time. For example, the compositions described herein can retain a 50%, 55%, 60%, 65%, 70%, 75%, 80%, 85%, 90%, 95% or more cannabinoid DE for extended periods of time.

By “thermodynamic stability”, it is meant that the compositions are in their lowest energy state, or in chemical equilibrium with their environment. This can be a dynamic equilibrium in which individual atoms or molecules change form, but their overall number in a particular form is conserved. Thermodynamic stability of compounds can be determined by measuring the enthalpy of formation (ΔHf) of individual compounds. The enthalpy of formation will be lesser if the compound is formed from its constituent elements enjoys some greater stability.

The composition can be at least 2-times more thermodynamically stable that a control composition that does not comprise an emulsifier. As illustrated in the Examples, the stability of the compositions described herein can be improved by including an emulsifier. For example, the compositions can be at least 2-times, at least 3-times, at least 4-times, at least 5-times or more, more thermodynamically stable than a control composition that does not comprise an emulsifier.

In some embodiments, a composition does not comprise a co-solvent. As used herein, the term “co-solvent” refers to a substance that can be added to a primary solvent in small amounts to increase the solubility of a poorly-soluble compound. Alcohols are frequently used as cosolvents in water (often less than 5% by volume) to dissolve hydrophobic molecules during extraction, screening, and formulation.

In an embodiment, compositions can comprise bioactive oils or encapsulated bioactive oils. Bioactive oils can be used for their pharmaceutical, cosmetic and nutritional properties. They can be volatile, sensitive to oxygen, light, moisture, and heat, which can diminish their applicability. Thus, encapsulation is one of the most efficient methods for the formulation of bioactive oils. The encapsulation system can be selected in line with the intended usage of the final formulation, which can vary depending on the size, shape or nature of the active components. In some embodiments, a composition does not comprise encapsulated oils or encapsulated bioactive oils.

Formulations

Compositions can be formulated as a powder, an effervescent powder, a tablet, an effervescent tablet, granules or effervescent granules.

Compositions described herein can be formulated, for example, by employing conventional vehicles or diluents, as well as additives of a type appropriate to the mode of desired administration (for example, excipients, preservatives, etc.) according to techniques known in the art of pharmaceutical formulation. The compositions can be formulated in a variety of unit dosage forms depending upon the method of administration. Suitable unit dosage forms, include, but are not limited to powders, tablets, pills, capsules, lozenges, sprays, granules, gums, etc.

Compositions can comprise: (i) about 20 to about 50 wt % alkaline agent (e.g., about 20, 25, 30, 40, 45, or 50 wt %), (ii) about 20 to about 50 wt % acidic agent (e.g., about 20, 25, 30, 40, 45, or 50 wt %), (iii) about 5 to about 40 wt % cyclodextrin (e.g., about 5, 10, or 15 wt %), (iv) about 1 to about 10 wt % cannabinoid (e.g., about 1, 2, 3, 4, 5, 6, 7, 8, 9, or 10 wt %), and (v) about 0 to about 10 wt % surfactant (e.g., 0.05 to 10 wt %, about 0, 0.5, 1, 2, 3, 4, 5, 6, 7, 8, 9, or 10 wt %). In some embodiment, the composition comprises (i) about 30 to about 40 wt % alkaline agent, (ii) about 40 to about 50 wt % acidic agent, (iii) about 10 to about 20 wt % cyclodextrin, (iv) about 1 to about 5 wt % cannabinoid, and (v) about 0 to about 5 wt % (e.g., 0.05 to 5 wt %) surfactant. In some embodiment, the composition comprises (i) about 32 to about 38 wt % alkaline agent, (ii) about 42 to about 48 wt % acidic agent, (iii) about 12 to about 16 wt % cyclodextrin, (iv) about 1 to about 4 wt % cannabinoid, and (v) about 0 to about 4 wt % (e.g., 0.05 to 4 wt %) surfactant.

For example, the composition can comprise (i) about 30 to about 40 wt % (e.g., about 30 to 35 wt %, about 35 to 40 wt %, about 30, 31, 32, 33, 34, 35, 36, 37, 38, 39, or 40 wt %) of a first alkaline agent, (ii) about 1 to about 10 wt % (e.g., about 1, 2, 4, 6, 8, or 10 wt %) of a second alkaline agent, (iii) about 40 to about 50 wt % acidic agent (e.g., about 40 to 45 wt %, about 45 to 50 wt %, about 40, 41, 42, 43, 44, 45, 46, 47, 48, 49, or 50 wt %), (iv) about 12 to about 20 wt % cyclodextrin (e.g., about 12, 14, 16, 18, or 20 wt %), (v) about 1 to about 4 wt % cannabinoid (e.g., about 1, 2, 3, or 4 wt %), and (vi) about 0 to about 5 wt % (e.g., 0.05 to 5 wt %, about 0, 0.5, 1, 2, 3, 4, or 5 wt %) surfactant. In some embodiments, the composition comprises (i) about 30 to about 35 wt % of a first alkaline agent, (ii) about 1 to about 6 wt % of a second alkaline agent, (iii) about 42 to about 48 wt % acidic agent, (iv) about 12 to about 16 wt % cyclodextrin, (v) about 2 to about 4 wt % cannabinoid, and (vi) about 0 to about 4 wt % (e.g., 0.05 to 4 wt %) surfactant. In some embodiments, the first alkaline agent is sodium bicarbonate, the second alkaline agent is potassium carbonate, and the acidic agent is citric acid.

In some embodiments, the composition comprises (i) about 20 to about 25 wt % sodium bicarbonate (e.g., about 20, 21, 22, 23, 24, or 25 wt %), (ii) about 1 to about 3 wt % (e.g., about 1, 2, or 3 wt %) potassium carbonate, (iii) about 30 to about 35 wt % citric acid (e.g., about 30, 31, 32, 33, 34, or 35 wt %), (iv) about 12 to about 20 wt % (e.g., about 12, 14, 16, 18, or 20 wt %) β-cyclodextrin, (v) about 5 to about 8 wt % (e.g., about 5, 6, 7, or 8 wt %) cannabinoid (e.g., delta-8 THC or THC), and (vi) about 0-5 wt % (e.g., about 0.1, 1, 2, 3, 4, or 5 wt %) Polysorbate 80. In some embodiments, the composition comprises (i) about 30 to about 36 wt % sodium bicarbonate, (ii) about 1 to about 6 wt % potassium carbonate, (iii) about 40 to about 50 wt % citric acid, (iv) about 10 to about 18 wt % β-cyclodextrin, (v) about 1 to about 5 wt % cannabinoid (e.g., delta-8 THC or THC), and (vi) about 0 to 5 wt % (e.g., 0.05 to 5 wt %) Polysorbate 80. In some embodiments, the composition comprises (i) about 30 to about 34 wt % sodium bicarbonate, (ii) about 1 to about 4 wt % potassium carbonate, (iii) about 42 to about 48 wt % citric acid, (iv) about 12 to about 16 wt % β-cyclodextrin, (v) about 1 to about 4 wt % cannabinoid (e.g., delta-8 THC or THC), and (vi) about 0 to 4 wt % (e.g., 0.05 to 4 wt %) Polysorbate 80.

The composition can comprise: (i) about 32 wt % sodium bicarbonate, (ii) about 3 wt % potassium carbonate, (iii) about 45 wt % citric acid, (iv) about 14 wt % ß-cyclodextrin, (v) about 2.5 wt % cannabinoid (e.g., delta-8 THC or THC), and (vi) from about 0 to 2 wt % (e.g., 0.05 to 2 wt %) Polysorbate 80. The balance of the composition can be any suitable a pharmaceutically acceptable carrier, diluent, or excipient.

In an aspect the composition can comprise (i) about 30, 31, 32, 33, or 34 wt % sodium bicarbonate, (ii) about 1, 2, 3, 4, 5, or 6 wt % potassium carbonate, (iii) about 42, 43, 44, 45, 46, 47, or 48 wt % citric acid, (iv) about 12, 13, 14, 15, or 16 wt % ß-cyclodextrin, (v) about 1, 2, 2.5, 3.0, 4, or 5 wt % cannabinoid (e.g., delta-8 THC or THC), and (vi) from about 0, 0.5, 1, 2, 3, or 4 wt % Polysorbate 80.

In certain embodiments, a composition is a recreational use composition. In certain embodiments, a composition is used for infusing a beverage or preparing a liquid concentrate. In certain embodiments, a composition is a pharmaceutical composition for medical use.

As used herein, the term “pharmaceutical composition” refers to a formulation including an active ingredient (e.g., cannabinoid), and optionally a pharmaceutically acceptable carrier, diluent, or excipient. The term “active ingredient” can interchangeably refer to an “effective ingredient” and is meant to refer to any agent that is capable of inducing a sought-after effect upon administration.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

The pharmaceutical compositions of this disclosure can be orally administered in any orally acceptable dosage form including, but not limited to, capsules, tablets, and aqueous suspensions and solutions. In the case of tablets for oral use, carriers, can include lactose, sorbitol, maltodextrin, dextrose, any sugar or sugar derivative, and corn starch. Lubricating agents, such as magnesium stearate, are also typically added. For oral administration in a capsule form, useful diluents include lactose and dried cornstarch. When aqueous suspensions and solutions and propylene glycol are administered orally, the active ingredient is combined with emulsifying and suspending agents. If desired, certain sweetening and/or flavoring and/or coloring agents can be added. Formulations described herein suitable for oral administration can be in the form of capsules, cachets, pills, tablets, lozenges (using a flavored basis, usually sucrose and acacia or tragacanth), powders, granules, or as a solution or a suspension in an aqueous or nonaqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compounds described herein can also be administered as a bolus, electuary or paste.

In solid dosage forms for oral administration (capsules, tablets, pills, dragees, powders, granules and the like), an active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions can also comprise buffering agents. Solid compositions of a similar type can also be employed as fillers in soft and hard-shelled gelatin capsules using such excipients as lactose or milk sugars, as well as high molecular weight polyethylene glycols and the like.

Tablets can be made by compression or molding, optionally with one or more accessory ingredients. Compressed tablets can be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets can be made in a suitable machine in which a mixture of the powdered compound is moistened with an inert liquid diluent. If a solid carrier is used, the preparation can be in tablet form, placed in a hard gelatin capsule in powder or pellet form, or in the form of a troche or lozenge. The amount of solid carrier will vary, e.g., from about 25 to 800 mg, preferably about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

Tablets and other solid dosage forms, such as dragees, capsules, pills, and granules, can optionally be scored or prepared with coatings and shells, such as enteric coatings and other coatings well known in the pharmaceutical-formulating art. They can alternatively or additionally be formulated so as to provide slow or controlled release of the active ingredient therein using, for example, hydroxypropyl methyl cellulose in varying proportions to provide the desired release profile, other polymer matrices, liposomes and/or microspheres. They can be formulated for rapid release, e.g., freeze-dried. They can be sterilized by, for example, filtration through a bacteria-retaining filter, or by incorporating sterilizing agents in the form of sterile solid compositions that can be dissolved in sterile water, or some other sterile injectable medium immediately before use. These compositions can also optionally contain opacifying agents and can be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Uses

Provided herein are methods of producing cannabinoid inclusion complexes dispersed in a solution comprising contacting any one of the compositions described herein with an aqueous solution.

An aqueous solution is a solution where the solvent is water. As used herein, “an aqueous solution” can refer to any solution that has water as a solvent, including for example any beverage, such as non-alcoholic beverages and alcoholic beverages. Non-limiting examples include tap water, mineral water, sparkling water, juices, soda, tea, coffee, milk, non-dairy milk, beer, wine, sparkling wine, liquor, and any combination thereof.

The cannabinoid inclusion complexes dispersed solution can comprise about 30-90% cannabinoid inclusion complexes. For example, the cannabinoid inclusion complexes dispersed solution can comprise about 30%, 40%, 50%, 60%, 70%, 80%, 90%, 95%, 99% or more cannabinoid inclusion complexes. The cannabinoid inclusion complexes dispersed in solution can comprise from about 0.01 to 5% of the final solution (e.g., 0.01, 0.02, 0.05, 0.1, 0.2, 0.3, 0.4, 0.5, 1, 1.5, 2, 2.5, 3, 3.5, 4, 4.5, or 5% of the final solution).

Contacting the composition and the aqueous solution can be without agitation or shaking. That is, a composition can be added to an aqueous solution and left with no mixing or movement such that dispersion of the composition is 50% or more complete.

Cannabinoids are highly hydrophobic so cannabinoid beverages can require special treatment through chemical or physical processes to achieve uniform distributions in aqueous solutions. For this reason, commercial cannabis beverages can be prepared as an emulsion or colloidal suspension of cannabinoids or cannabinoid-containing additives, however, such compositions (except for micro-emulsions) are thermodynamically unstable. Cannabinoid-containing beverages can require mixing or shaking before opening. Mixing a premade beverage prior to consumption is undesirable to consumers, and if the beverage is carbonated can be of greater concern. Excessive dissolved carbon dioxide gas released upon opening a container can burst the liquid from its container and render the beverage flat, which is typically perceived as low quality.

Liquid concentrates present an attractive option by which to infuse cannabinoids into a beverage with fewer concerns for shear mixing or maintaining uniformity over a long period of time as seen with ready-to-go cannabinoid beverage solutions or dispersions. However, liquid concentrates require liquid-proof packaging and those which are not in a single-use packaging cannot economically control dosing via volumetric dispensing. Additionally, liquid concentrates require a significant concentration of surfactants and cosolvents to maintain solution stability, which can be undesirable to consumers. Lastly, aqueous environments can be susceptible to rancidity and/or bacterial growth making the commercialization process more costly.

The compositions described herein have enhanced dispersibility properties, such that the composition can reach dispersion goal (e.g., 50%, 60%, 70%, 80%, 90% or more dispersion) simply by contacting the composition with the aqueous solution (e.g., by dropping a tablet or powder form of the composition into the aqueous solution). The contacting can require no agitation, shaking, stirring or the like of the solution to reach such dispersion percent. In some embodiments however, adding a step of agitating, shaking, or stirring the solution can be used to increase further the dispersibility of the composition.

Contacting a composition with an aqueous solution can be done at a low temperature. For example, the contacting can occur at a temperature as low as 20, 15, 10, 5, or 1° C. such that dispersion of the composition is 50% or more complete.

Compositions described herein have enhanced dispersibility properties, such that the composition can reach dispersion goal (e.g., 50% or more dispersion) by contacting the composition with an aqueous solution regardless of the temperature of the aqueous composition (e.g., the aqueous composition can be cold, at room temperature or hot). For example, the contacting can be at a temperature ranging from about 1° C. to 100° C. The contacting can require no agitation, shaking, stirring or the like of the solution to reach such dispersion percent. In some embodiments adding a step of agitating, shaking, or stirring the solution can be used to increase further the dispersibility of the composition.

An additional embodiment provides a method of making a cannabinoid-infused food product or beverage comprising: (i) producing cannabinoid inclusion complexes dispersed in a solution by contacting any one of the compositions described herein with an aqueous solution, and (ii) incorporating the cannabinoid inclusion complexes dispersed in a solution of (i) in a food product or in a beverage, thereby making a cannabinoid-infused food product or beverage.

The food product or beverage can be, for example, jellies, edibles, non-alcoholic beverages, and alcoholic beverages.

As used herein, the term “edible” refers to any edible products such as gummies, candy, cookies, brownies, and other food or drinks that contain a cannabinoid.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1

### Obtaining Cannabinoid Cyclodextrin Inclusion Complexes

A beta-cyclodextrin inclusion complex of delta-8 THC was obtained from a commercial vendor with a delta-8 THC concentration of approximately 16.6% by weight and a beta-cyclodextrin concentration of approximately 83.4% by weight.

Alternatively, a beta-cyclodextrin inclusion complex of delta-8 THC was prepared via the ethanoic solution method. Into a single-necked 500 mL round-bottomed flask was charged beta-cyclodextrin (83.5 g), ethanol (90 mL), water (10 mL), and delta-8 THC (16.5 g) and the mixture was refluxed under nitrogen for 12 hours. The resulting solution concentrated using a rotary evaporator to obtain a dry, white powder.

Alternatively, A beta-cyclodextrin inclusion complex of delta-8 THC was prepared via the precipitation complexation method by Mannila et al. (2007).

The delta-8 THC oil was incorporated via an inclusion complex of ß-cyclodextrin using the techniques described above. The resulting free-flowing powder was blended a powdered effervescing agent and surfactant to produce the effervescent composition described herein.

### Example 2

### Analysis of the Cannabinoid Cyclodextrin Inclusion Complexes Dispersion

The ideal solvent for cannabinoid cyclodextrin inclusion complexes was determined by measuring absorbance spectra for various ethanol/water cosolvent systems. A lambda max value was determined for delta-8 THC cyclodextrin inclusion complexes at 275 nm and 282 nm.

Ultraviolet-visible (UV-visible) spectrophotometry is primarily a quantitative analytical technique concerned with the absorption of near-UV (180-390 nm) or visible (390-780 nm) radiation by chemical species in solution. These regions of the electromagnetic spectrum provide energy that gives rise to electronic transitions. Under controlled experimental conditions the amount of radiation absorbed can be directly related to the concentration of the analyte in solution. The relationship between absorbance and concentration is known as Beer's law (also referred to by other names such as the Beer-Lambert law and the Bouguer-Lambert-Beer law) and is defined by the equation:

A=εbc

where A is the absorbance of the solution (no units), E is the molar absorptivity (units of l·mol⁻¹cm⁻¹), b is the path length of radiation through the absorbing medium (units of cm), and c is the concentration (units of mol·l⁻¹). UV-Vis spectrophotometry is a well know method to quantify both organic (primarily in the near-UV) and inorganic (primarily in the visible) species.

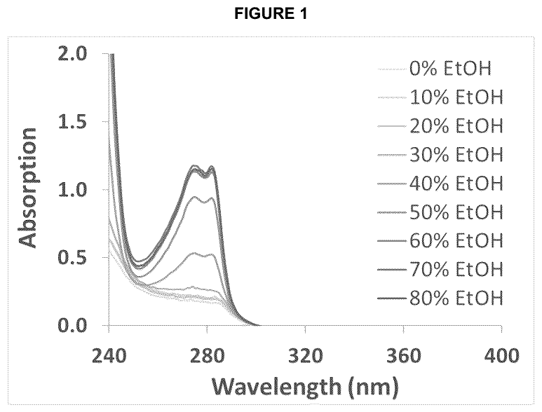

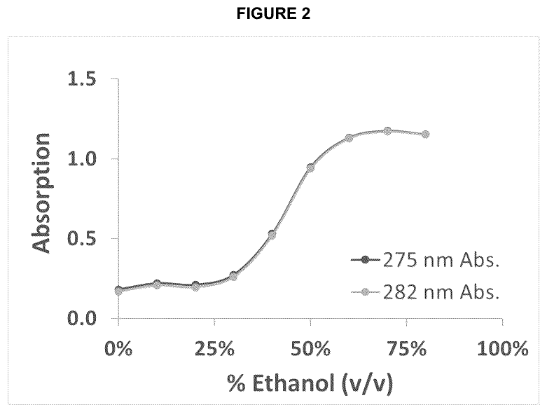

The absorption spectra for delta-8 THC cyclodextrin inclusion complex dispersed in various ethanol/water cosolvents (including from 0 to 80% ethanol) was measured and is shown in FIG. 1. The lambda max absorption value for various ethanol concentrations is shown in FIG. 2. Solutions containing greater than 80% ethanol by volume were not analyzed due to precipitation in solution.

### Example 3

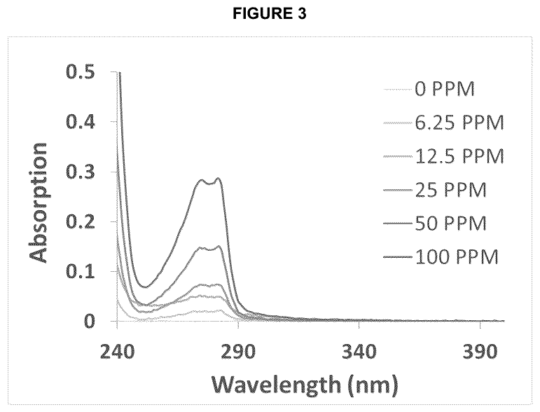

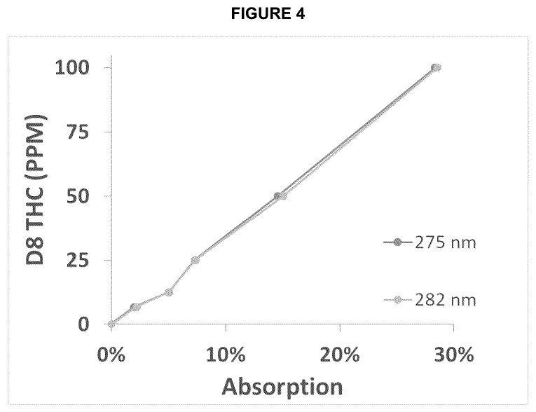

A 6-point calibration curve was obtained in a concentration range of 0 ppm to 100 ppm of delta-8 THC beta-cyclodextrin inclusion complex. A 100 ppm stock solution of delta-8 THC in a cyclodextrin inclusion complex was prepared in a 60/40 v/v ethanol/water mixture that was heated at 45° C. with vigorous mixing for 1 hour. As seen in FIG. 3, which illustrates UV/Vis spectrophotometry calibration curve obtained from 0, 6.25, 12.5, 25, 50, and 100 ppm delta-8 THC concentrations, a 6-point calibration curve that obeys with Beer's law was obtained within 99% regression. FIG. 4 shows the UV/Vis spectra obtained from the 6-point calibration method.

### Example 4

### Illustrative Compositions of Effervescent Cannabinoid Cyclodextrin Inclusion Complexes

Table 1 illustrate illustrative compositions used to make the tablets or granules of the present disclosure, using various dosage of surfactant.

TABLE 1
Illustrative examples of effervescent tablet compositions.

| | Entry | Entry | Entry | Entry | Entry | Entry | Entry |
|---|---|---|---|---|---|---|---|
| Ingredient | 1 | 2 | 3 | 4 | 5 | 6 | 7 |
| Sodium Bicarbonate | 43.2 | 43.2 | 43.2 | 43.2 | 43.2 | 43.2 | 43.2 |
| Potassium Carbonate | 4.8 | 4.8 | 4.8 | 4.8 | 4.8 | 4.8 | 4.8 |
| Citric Acid | 40.3 | 40.3 | 40.3 | 40.3 | 40.3 | 40.3 | 40.3 |
| β-cyclodextrin | 9.8 | 9.8 | 9.8 | 9.8 | 9.8 | 9.8 | 9.8 |
| delta-8 THC | 1.9 | 1.9 | 1.9 | 1.9 | 1.9 | 1.9 | 1.9 |
| Polysorbate 80 | 0.0 | 0.05 | 0.1 | 0.25 | 0.5 | 1.0 | 2.0 |

The delta-8 THC oil was incorporated via an inclusion complex of ß-cyclodextrin using the techniques described in Example 1. The resulting free-flowing powder was blended with a powdered effervescing agent and surfactant to produce the effervescent composition. A 22.5 mg strength delta-8 THC tablet having a total tablet weight of about 1200 mg is comprised of sodium bicarbonate, potassium carbonate, citric acid, beta-cyclodextrin, delta-8 THC, and polysorbate. These compositions were used for testing bCD complex dispersion in an aqueous solution and corresponding solution/dispersion stability over time.

The tablet was prepared by blending the powder mixture in a ribbon blender. In entries containing polysorbate 80 the bCD complex addition occurred after all other ingredients were uniformly mixed. The blend was directly compressed on a tablet press using 13 mm, standard concave tooling into a tablet for beverage infusion. The 1200 mg tablet was allowed to effervesce in a 500 mL solution of deionized water at 23 C. After 10 minutes the solution was inverted 3 times and the dispersion efficiency for the targeted cannabinoid was measured. Each solution was stored for 45 days at 23 C to monitor dispersion stability for the target cannabinoid.

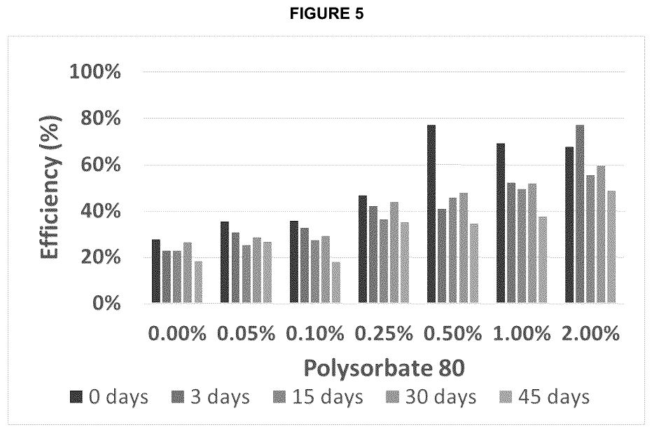

As illustrated in FIG. 5 the percent of added beta-cyclodextrin inclusion complex dispersed in solution from an effervescent composition was modulated by the percent of added polysorbate.

The general procedure was to add a 1.2 g tablet of the compositions described in Table 1 to a 500-mL solution of deionized water. After approximately 5 minutes the bottle was inverted 3× and a 1.2 mL aliquot of the solution was dispensed into 1.8 mL of ethanol for UV/VIS spectrometry analysis. The resulting solutions illustrated that the addition of surfactant in the composition unexpectedly increased dissolved delta-8 THC in solution by at least 2×, or up to 3× than when no surfactant was present.

As illustrated in FIG. 5 and Table 2, which show the stability of delta-8 THC in solutions prepared from the effervescent compositions, the delta-8 THC concentration remained relatively stable over a period of 45 days, with those compositions containing surfactant exhibited improved stability, by at least 2×, or up to 3× the control.

TABLE 2
Stability of delta-8 THC in solutions prepared from the
effervescent compositions.

| | Dispersion Efficiency | | | | | | |
|---|---|---|---|---|---|---|---|
| | Tween 80 Weight % | | | | | | |
| Shelf | 0.00% | 0.05% | 0.10% | 0.25% | 0.50% | 1.00% | 2.00% |
| Life | Entry | Entry | Entry | Entry | Entry | Entry | Entry |
| (days) | 1 | 2 | 3 | 4 | 5 | 6 | 7 |
| 0 days | 28% | 35% | 36% | 47% | 77% | 69% | 68% |
| 3 days | 23% | 30% | 33% | 42% | 41% | 52% | 77% |
| 15 days | 23% | 25% | 27% | 36% | 46% | 49% | 55% |
| 30 days | 26% | 28% | 29% | 44% | 48% | 52% | 59% |
| 45 days | 18% | 27% | 18% | 35% | 35% | 37% | 49% |

### Example 5

### Illustrative Compositions of Effervescent Cannabinoid Compositions without Inclusion Complexes

Table 3 illustrate illustrative compositions used to make the tablets or granules of an alternate composition, using various dosage of surfactant.

TABLE 3
Illustrative examples of effervescent tablet compositions.

| | Entry | Entry | Entry | Entry | Entry | Entry | Entry |
|---|---|---|---|---|---|---|---|
| Ingredient | 1 | 2 | 3 | 4 | 5 | 6 | 7 |
| Sodium Bicarbonate | 43.2 | 43.2 | 43.2 | 43.2 | 43.2 | 43.2 | 43.2 |
| Potassium Carbonate | 4.8 | 4.8 | 4.8 | 4.8 | 4.8 | 4.8 | 4.8 |
| Citric Acid | 40.3 | 40.3 | 40.3 | 40.3 | 40.3 | 40.3 | 40.3 |
| β-cyclodextrin | 0.0 | 0.0 | 0.0 | 0.0 | 0.0 | 0.0 | 0.0 |
| delta-8 THC | 1.9 | 1.9 | 1.9 | 1.9 | 1.9 | 1.9 | 1.9 |
| Polysorbate 80 | 0.0 | 0.05 | 0.1 | 0.25 | 0.5 | 1.0 | 2.0 |

The delta-8 THC oil was incorporated via ethanol infusion on sodium bicarbonate by mixing the measured amount of delta-8 THC with anhydrous ethanol, mixing the solution with bicarbonate powder, and evaporating to dryness. The resulting free-flowing powder was blended with a powdered effervescing agent and surfactant to produce the effervescent composition. A 22.5 mg strength delta-8 THC tablet having a total tablet weight of about 1200 mg is comprised of sodium bicarbonate, potassium carbonate, citric acid, beta-cyclodextrin, delta-8 THC, and polysorbate. These compositions were used for testing delta-8 THC dispersion in an aqueous solution.

The tablet was prepared by blending the powder mixture in a ribbon blender. In entries containing polysorbate 80 the surfactant was added after all other ingredients were uniformly mixed. The blend was directly compressed on a tablet press using 13 mm, standard concave tooling into a tablet for beverage infusion. The 1200 mg tablet was allowed to effervesce in a 500 mL solution of deionized water at 23 C. After 10 minutes the solution was inverted 3 times and the dispersion efficiency for the targeted cannabinoid was measured.

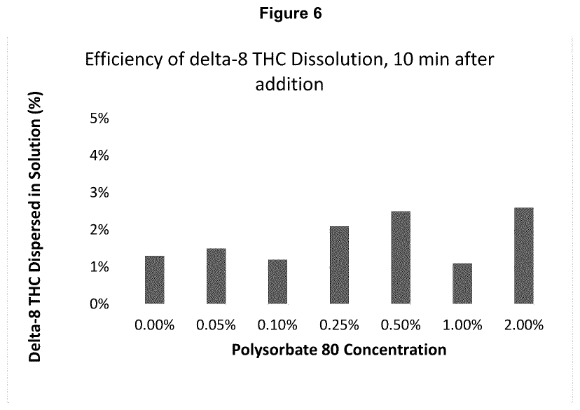

As illustrated in FIG. 6, the percent of delta-8 THC dispersed in solution from the effervescent composition was not modulated by the percent of added polysorbate, but the overall dispersion efficiency was greatly reduced as compared to beta-cyclodextrin compositions.

The general procedure was to add a 1.2 g tablet of the compositions described in Table 3 to a 500-mL solution of deionized water. After approximately 5 minutes the bottle was inverted 3× and a 1.2 mL aliquot of the solution was dispensed into 1.8 mL of ethanol for UV/VIS spectrometry analysis. The resulting solutions illustrated that the addition of surfactant in the composition had a negligible effect on dissolved delta-8 THC in solution, and only a fraction of the amount observed with the beta-cyclodextrin inclusion complex.

### Example 6

### Maltodextrin/Effervescent Agent Ratio

A 22.5 mg strength delta-8 THC tablet having a total tablet weight of about 1200 mg comprising of sodium bicarbonate, potassium carbonate, citric acid, maltodextrin, beta-cyclodextrin, delta-8 THC, and polysorbate was generated. Exemplary formulations manufactured for this embodiment are provided in Table 4. These compositions were used for testing bCD complex dispersion in an aqueous solution in agitated and non-agitated modes. For a non-agitated test a 1200 mg tablet was placed 500 mL of deionized water and after 1 hour and no agitation of the solution the dispersion efficiency was measured. The dispersions were mixed at 500 RPM for 1 hour and the dispersion efficiency measured.

TABLE 4
Effervescent tablet compositions.
Effervescing Agent Content

| | Entry | Entry | Entry | Entry | Entry | Entry | Entry | Entry |
|---|---|---|---|---|---|---|---|---|
| Ingredient | 14 | 15 | 16 | 17 | 18 | 19 | 20 | 21 |
| Sodium | 4.9 | 9.8 | 14.7 | 19.6 | 24.5 | 29.4 | 34.3 | 39.1 |
| Bicarbonate | | | | | | | | |
| Potassium | 0.5 | 1.1 | 1.6 | 2.2 | 2.7 | 3.3 | 3.8 | 4.3 |
| Carbonate | | | | | | | | |
| Citric Acid | 4.6 | 9.1 | 13.7 | 18.3 | 22.8 | 27.4 | 31.9 | 36.5 |
| Effervescing | 10.0 | 20.0 | 30.0 | 40.0 | 50.0 | 60.0 | 70.0 | 80.0 |
| Agent(s) | | | | | | | | |
| Maltodextrin | 77.3 | 67.3 | 57.3 | 47.3 | 37.3 | 27.3 | 17.3 | 7.3 |
| β-cyclodextrin | 9.8 | 9.8 | 9.8 | 9.8 | 9.8 | 9.8 | 9.8 | 9.8 |
| delta-8 THC | 1.9 | 1.9 | 1.9 | 1.9 | 1.9 | 1.9 | 1.9 | 1.9 |
| Polysorbate 80 | 1.0 | 1.0 | 1.0 | 1.0 | 1.0 | 1.0 | 1.0 | 1.0 |

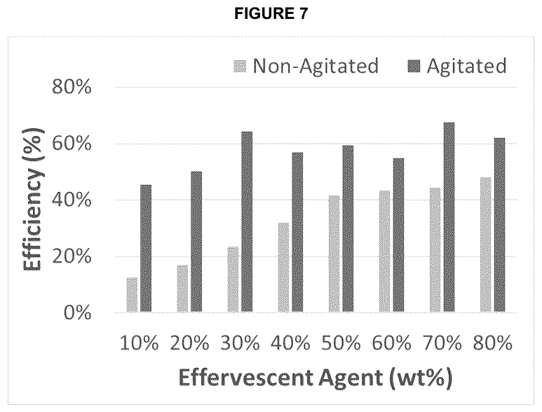

FIG. 7 and Table 5 provide the dispersibility results obtained. It was for example demonstrated that increasing effervescing agent amount in the composition was associated with dispersion enhancement for non-agitated solutions, and that at least 50% effervescing agent was needed to achieve desirable result. It was also found that 65-85% effervescing agent was most desirable based on dispersion efficiency and time to disperse (complete dispersion in 4-8 minutes, which is desirable for a dispersion time in a drink).

TABLE 5
Dispersion efficiency for various effervescing agent
concentrations in agitated and non-agitated solution.
Dispersion times in minutes per gram.

| | Dispersion Efficiency & Time to Disperse | | | | | | | |
|---|---|---|---|---|---|---|---|---|
| | Effervescence Content % | | | | | | | |
| | 10% | 20% | 30% | 40% | 50% | 60% | 70% | 80% |
| Non-Agitated | 12% | 17% | 23% | 32% | 42% | 43% | 44% | 48% |
| Agitated | 45% | 50% | 64% | 57% | 59% | 55% | 67% | 62% |
| Time to Disperse | 58 | 50 | 28 | 22 | 15 | 13 | 9 | 5 |
| (min/g) | | | | | | | | |

Although the present disclosure has been described with reference to specific details of certain embodiments thereof in the above examples, it will be understood that modifications and variations are encompassed within the spirit and scope of the disclosure. Accordingly, the methods and compositions are limited only by the following claims.

What is claimed is:

1. A composition comprising:
(i) from about 20 to 95 wt % of an effervescing agent;
(ii) from about 5 to 80 wt % of a cannabinoid cyclodextrin inclusion complex, and
(iii) from about 0 to 5 wt % of an emulsifier,
wherein the cannabinoid cyclodextrin inclusion complex comprises a cyclodextrin host and a cannabinoid guest.

2. The composition of claim 1, wherein the effervescing agent comprises an acidic agent and an alkaline agent, wherein the effervescing agent is present at 50-85 wt % of the composition.

3. The composition of claim 2, wherein the acidic agent is citric acid, ascorbic acid, or a combination thereof.

4. The composition of claim 2, wherein the alkaline agent is a sodium salt of bicarbonate, a potassium salt of bicarbonate, a sodium salt of carbonate, a potassium salt of carbonate or any combination thereof.

5. The composition of claim 1, wherein the cannabinoid guest is delta-8 tetrahydrocannabinol (THC), delta-9 THC, delta-10 THC, cannabidiol (CBD), cannabinol (CBN), cannabigerol (CBG), hexahydrocannibinol (HHC), tetrahydrocannabivarin (THCV), tetrahydrocannabiphorol (THCP), 0-acetate thereof, isomers thereof, or combinations thereof.

6. The composition of claim 1, wherein the cyclodextrin host is alpha-cyclodextrin, beta-cyclodextrin, gamma-cyclodextrin, 2-Hydroxypropyl-β-cyclodextrin, β-cyclodextrin sulfobutyl ether, or combinations thereof.

7. The composition of claim 1, further comprising one or more additives.

8. The composition of claim 7, wherein the composition comprises about 5-25 wt % of the one or more additives.

9. The composition of claim 1, wherein the emulsifier is present at from about 0.5 to 5 wt %.

10. The composition of claim 9, wherein the emulsifier is a natural emulsifier or polysorbate 80.

11. The composition of claim 7, wherein the one or more additives are selected from the group consisting of terpenes, terpenoids, flavonoids, excipients, antioxidants, sweeteners, lubricants, colorants, vitamins, disintegrants, defoamers, foaming agents, wax, bitter blockers, and combinations thereof.

12. The composition of claim 1, wherein the cannabinoid cyclodextrin inclusion complex comprises about 5-25 wt % cannabinoid and about 75-95 wt % cyclodextrin.

13. The composition of claim 12, wherein the cannabinoid cyclodextrin inclusion complex comprises about 16 wt % of cannabinoid and about 84 wt % cyclodextrin.

14. The composition of claim 1, wherein the composition has a dispersibility ranging from about 50 to 95%.

15. The composition of claim 1, wherein the composition has a shelf-life of at least about 45 days.

16. The composition of claim 1, wherein the composition has at least 2-times more dispersity than a control composition that does not comprise an emulsifier.

17. The composition of claim 1, formulated as a powder, an effervescent powder, a tablet, an effervescent tablet, granules or effervescent granules.

18. The composition of claim 1 comprising:
(i) about 32 wt % sodium bicarbonate,
(ii) about 3 wt % potassium carbonate,
(iii) about 45 wt % citric acid,
(iv) about 14 wt % ß-cyclodextrin,
(v) about 2.5 wt % cannabinoid,
(vi) from about 0 to 2 wt % Polysorbate 80.

19. The composition of claim 18, wherein the wherein the cannabinoid is delta-8 tetrahydrocannabinol (THC), delta-9 THC, delta-10 THC, cannabidiol (CBD), cannabinol (CBN), cannabigerol (CBG), hexahydrocannibinol (HHC), tetrahydrocannabivarin (THCV), tetrahydrocannabiphorol (THCP), 0-acetate thereof, isomers thereof, or combinations thereof.

20. A method of producing cannabinoid inclusion complexes dispersed in a solution comprising contacting the composition of claim 1 with an aqueous solution,
wherein the cannabinoid inclusion complexes dispersed solution comprises at least 50% cannabinoid inclusion complexes,
thereby producing cannabinoid inclusion complexes dispersed in a solution.

21. The method of claim 20, wherein contacting the composition and the aqueous solution is without agitation or shaking.

22. The method of claim 20, wherein contacting the composition and the aqueous solution comprises contacting at a temperature as low as 1° C.

23. A method of making a cannabinoid-infused food product or beverage comprising:
(i) producing cannabinoid inclusion complexes dispersed in a solution by contacting the composition of claim 1 with an aqueous solution, and
(ii) incorporating the cannabinoid inclusion complexes dispersed in a solution of (i) in a food product or in a beverage,
thereby making a cannabinoid-infused food product or beverage.

24. The method of claim 23, wherein the food product or beverage is jellies, edibles, non-alcoholic beverages, or alcoholic beverages.

25. The method of claim 23, wherein contacting the composition and the aqueous solution is without agitation or shaking.

26. The method of claim 23, wherein contacting the composition and the aqueous solution comprises contacting at a temperature lower than 20° C.
